(19) United States
(12) Patent Application Publication
(10) Pub. No.: US 2008/0145937 A1
(43) Pub. Date: Jun. 19, 2008

(54) In Vivo Transformation of Pancreatic Acinar Cells into Insulin-Producing Cells

(75) Inventors: Paul A. Grayburn, DeSoto, TX (US); Shuyuan Chen, Allen, TX (US)

Correspondence Address: CHALKER FLORES, LLP, 2711 LBJ FRWY, Suite 1036, DALLAS, TX, 75234, US

(73) Assignee: BAYLOR RESEARCH INSTITUTE, Dallas, TX (US)

(21) Appl. No.: 11/859,709

(22) Filed: Sep. 21, 2007

Related U.S. Application Data
(60) Provisional application No. 60/846,465, filed on Sep. 22, 2006.

(51) Int. Cl. C12N 15/87; C12N 15/00; C12N 5/00

(52) U.S. Cl. 435/459; 435/455; 435/461; 435/465; 435/320.1; 435/325

(57) ABSTRACT

The present invention includes compositions and methods for transforming cells into glucose-responsive, insulin-production cells using a construct that expresses betacellulin and PDX1, e.g., transforming pancreatic acinar cells using one or more expression vectors that expressed betacellulin and PDX1 using ultrasound targeted microbubble destruction (UTMD).

### CROSS-REFERENCE TO RELATED APPLICATIONS

This application claims priority to U.S. Provisional Application Ser. No. 60/846,465, filed Sep. 22, 2006, the entire contents of which are incorporated herein by reference.

### STATEMENT OF FEDERALLY FUNDED RESEARCH

This invention was made with U.S. Government support under Contract No. P01 DK58398 awarded by the NIH. The government has certain rights in this invention.

### TECHNICAL FIELD OF THE INVENTION

The present invention relates to treatments for diabetes, and more particularly, to compositions and methods for the transformation of cells into glucose-responsive, insulin producing cells.

### BACKGROUND OF THE INVENTION

Without limiting the scope of the invention, its background is described with respect to diabetes.

Diabetes affects approximately 200 million people worldwide and is increasing in prevalence (1). It is estimated to be the fifth leading cause of death in the world (2), and results in serious complications, including cardiovascular disease, chronic kidney disease, blindness, and neuropathy.

Despite a wide variety of pharmacological treatments for diabetes, including insulin therapy, adequate blood sugar control is often difficult, in part because these agents are not able to duplicate the glucose regulatory function of normal islets. Accordingly, new treatment strategies have focused on replenishing the deficiency of beta cell mass common to both major forms of diabetes by either islet transplantation or beta-cell regeneration (3-5).

One promising opportunity in the area of diabetes therapy is taught by U.S. Pat. No. 6,232,288, issued to Kojima for composition for improving pancreatic function. Kojima teaches the use of the betacellulin protein itself, or a fragment thereof, to promote the differentiation of undifferentiated pancreatic cells into insulin-producing beta cells or pancreatic polypeptide producing F cells. The BTC protein composition improved glucose tolerance in patients and inhibited the growth of undifferentiated pancreatic cells. Methods for treating mammals, including humans, were also provided, however, long-term treatment of the diabetic condition was not achieved by providing the patient with the betacellulin protein intravenously.

The betacellulin protein (BTC) is a peptide factor produced by pancreatic beta tumor cells derived from a transgenic mouse, and its full amino acid sequence has been clarified by cDNA analysis (Shing et al., Science, 259:1604 (1993); Sasada et al., Biochemical and Biophysical Research Communications, 190:1173 (1993)). The mRNA of BTC has been detected in non-brain organs, e.g., liver, kidney and pancreas, suggesting that BTC protein may exhibit some function in these organs. BTC protein was first discovered as a factor possessing mouse 3T3 cell growth-promoting activity and was later found to exhibit growth-promoting activity against vascular smooth muscle cells and retinal pigment epithelial cells (Shing et al., Science, 259:1604 (1993)).

The human BTC protein occurs naturally in very trace amounts. Highly purified human BTC protein was produced recombinantly in large amounts and at relatively low costs (EP-A-0555785). Two other patent applications (EP-A-0482623, EP-A-0555785) indicate that the BTC protein can be used in the treatment of diseases such as wounds, tumors and vascular malformations, and preparation of competitive agents such as an antibodies or false peptides which can be used in the treatment of such diseases attributable to smooth muscle growth as atherosclerosis and diabetic retinopathy. Despite the availability of these reagents, a great need still exists for compositions and methods for the long-term treatment of diabetes.

### SUMMARY OF THE INVENTION

The present invention uses compositions and methods for the transformation of cells into glucose-responsive, insulin-producing cells using betacellulin and Pancreas Duodenum Homeobox-1 (PDX1). In one specific example, pancreatic acinar cells may be transformed in vivo using ultrasound targeted microbubble destruction (UTMD) with one or more expression vectors that delivery betacellulin and Pancreas Duodenum Homeobox-1 (PDX1) genes to a target or host cell.

More particularly, the present invention includes compositions and methods for inducing insulin production in cells by transforming one or more cells with a construct that expresses betacellulin (BTC) and Pancreas Duodenum Homeobox-1 (PDX1), e.g., the cells are transformed with a construct that co-expresses PDX1 and BTC. In one embodiment the cells are selected from pancreatic islet cells, pancreatic acinar cells, cell lines, cells that have been co-transfected with one or more insulin genes. The construct may be delivered using microbubbles, calcium phosphate-DNA co-precipitation, DEAE-dextran-mediated transfection, polybrene-mediated transfection, electroporation, microinjection, liposome fusion, lipofection, protoplast fusion, retroviral infection, and biolistics or other methods as will be known to those of skill in the art. In one specific embodiment, the construct is delivered using microbubbles and ultrasound targeted microbubble destruction.

It has been found that using the compositions and methods of the present invention, pancreatic cells were able to express insulin based on glucose levels for more than 15 days, that is, the cells became glucose-responsive, insulin-producing cells. The cells can be transformed in vivo and express insulin in a glucose responsive manner for more than 15 days. In one example, the cells are nonendocrine pancreas cells are express insulin in a glucose responsive manner for more than 15 days. The target cells may be rendered glucose-responsive, insulin-producing by expression of BTC selected from mouse, rat or human BTC and PDX1 is selected from mouse, rat or human PDX1.

The present invention also includes a vector having a nucleic acid expression construct that expresses betacellulin or PDX1 or both when transfected into acinar cells. The skilled artisan will recognize that a variety of methods of delivering the vector of the present invention may be used. Non-limiting examples of delivery include precipitation (e.g., Calcium phoshohate), liposomal, electroporation and projectile. The vector may be delivered in a microbubble that is destroyed upon exposure to ultrasound to target selected cells or tissues. In one embodiment the vector is a nucleic acid expression construct that includes a promoter the controls the expression of BTC. When expressed along with PDX1, the nucleic acid expression construct may have BTC and PDX1 under the control of the same promoter. Alternatively, the BTC and PDX1 may be on separate vectors and even under the control of different promoters. Non-limiting examples of promoters for use with the present invention include rat insulin promoter (RIP), Human Immunodeficiency Virus (HIV), avian myeloblastosis virus (AMV), SV40, Mouse Mammary Tumor Virus (MMTV) promoter, Human Immunodeficiency Virus Long Terminal Repeat (HIV LTR) promoter, Moloney virus promoter, avian leukosis virus (ALV) promoter, Cytomegalovirus (CMV) promoter, human Actin promoter, human Myosin promoter, RSV promoter, human Hemoglobin promoter, human muscle creatine promoter and EBV promoter. BTC for expression using the present invention may be a mammalian BTC, e.g., mouse, rat or human BTC. PDX1 for expression using the present invention may be a mammalian PDX1, e.g., mouse, rat or human PDX1. In one specific example the PDX1 is Genbank Accession No. NM022852 and the BTC is Genbank Accession No. NM022256.

The present invention also includes host cells that include an exogenous nucleic acid segment that expresses BTC and PDX1 under the control of a constitutive promoter. The host cell may also have an exogenous nucleic acid segment that expresses BTC and PDX1 under the control of a constitutive promoter. The BTC and/or PDX1 gene may be under the control of a promoter is selected from, e.g., RIP, HIV, AMV, SV40, Mouse Mammary Tumor Virus (MMTV) promoter, Human Immunodeficiency Virus Long Terminal Repeat (HIV LTR) promoter, Moloney virus promoter, ALV promoter, Cytomegalovirus (CMV) promoter, human Actin promoter, human Myosin promoter, RSV promoter, human Hemoglobin promoter, human muscle creatine promoter and EBV promoter. Examples of host cells include, but are not limited to, pancreatic islet cells, pancreatic acinar cells, primary pancreatic cells, or other cells that have been co-transfected with one or more insulin genes. The host cell may be transformed by microbubbles, calcium phosphate-DNA co-precipitation, DEAE-dextran-mediated transfection, polybrene-mediated transfection, electroporation, microinjection, liposome fusion, lipofection, protoplast fusion, retroviral infection, and biolistics. In one example, when the target cells are transformed in vivo the cells may express one or more pancreatic beta cell markers, e.g., INS-1, INS-2, glucagon, somatostatin, MIST-1, VMAT, neurogenin-3, Nkx2.2 and combinations thereof.

### BRIEF DESCRIPTION OF THE DRAWINGS

The accompanying figures, in which like reference numerals refer to identical or functionally-similar elements throughout the separate views and which are incorporated in and form part of the specification, further illustrate the present invention and, together with the detailed description of the invention, serve to explain the principles of the present invention.

FIG. 1. Top Panel. Plot of serum glucose levels over time. In normal controls (solid red line), glucose levels are stable. In all Streptozotocin (STZ)-treated rats, glucose rises precipitously by day 3, and continues to rise in rats treated with STZ only (solid black line), DsRed (dashed black line) or betacellulin alone (dashed blue line). Glucose improves by day 5 and 10 in rats treated with Pancreas Duodenum Homeobox-1 (PDX1) (dashed orange line), and is nearly normal in rats treated with both PDX1 and betacellulin (solid blue line). By repeated measures ANOVA, these differences are highly statistically significant both between groups and over time. Bottom Panel. Plot of serum insulin levels over time. In normal controls (red line), insulin levels are stable over time. In all STZ-treated rats, insulin declines significantly by day 3, and either decline further (STZ only or UTMD with DsRed), or stay low (UTMD with betacellulin). In contrast, UTMD with PDX1 restores insulin levels to normal by day 5 (dashed orange line); whereas UTMD with PDX1 and betacellulin results in supranormal insulin levels at days 5 and 10 (solid blue line). By repeated measures ANOVA, these differences are highly statistically significant both between groups and over time.

FIG. 2. Plot of C-peptide at baseline and day 10. In normal controls, C-peptide is stable over time (red lines). C-peptide decreases in rats treated STZ alone or STZ followed by UTMD with DsRed or BTC. However, C-peptide increases significantly in rats treated with STZ followed by UTMD with BTC and PDX1 (p<0.03 vs all other groups at day 10).

FIG. 3. Results of a glucose tolerance test performed 10 days after UTMD. The rats treated with STZ alone (black line) have markedly elevated blood glucose levels at baseline that increase further after glucose challenge. The betacellulin and PDX1 (blue line) have a nearly normal glucose response that is similar to normal control rats (red line).

FIG. 4. Representative histological sections of rat pancreas stained with FITC-labeled anti-insulin (green) and CY5-labeled anti-glucagon (blue) antibodies. Left upper panel. Low power (100×) section from a normal control rat showing 3 typical islets with beta-cells in the center (green) and alpha-cells on the periphery (blue). Right upper panel. Low power section (100×) from a STZ-treated rat showing no visible islets. Left middle panel. Low power section (100×) from a BTC-treated rat showing atypical islet-like clusters of cells that stain mostly with glucagons (blue). Right middle panel. Low power section (100×) from a rat treated with PDX1 and betacellulin plasmids by UTMD. Atypical islet-like clusters of cells stain mostly with glucagons. In addition, anti-insulin appears to be present diffusely throughout the exocrine pancreas. Left lower panel. Higher power (400×) image from a rat treated with PDX1 and betacellulin plasmids showing prominent insulin staining in what appear to be acinar cells. Right lower panel. Higher power (400×) image of the atypical islet-like clusters in a rat treated with BTC and PDX1. Glucagon staining is predominant in these islet-like clusters.

FIG. 5. Plot showing number of islets per slide for normal islet morphology (left panel) and islet-like clusters of predominantly glucagon-positive cells (right panel). Normal islets were common in controls (46±9 islets per slide), but rare in STZ-rats, regardless of treatment group (p<0.0001). Islet-like clusters of glucagons-positive cells were absent in controls, present in modest numbers in STZ-rats, particularly in those treated with both BTC and PDX1 (19±8 per slide, p<0.02 vs other groups).

FIG. 6. Left Panels. High power images (1000×) from a rat treated with PDX1 and betacellulin by UTMD. The top left image is stained with FITC-labeled anti-insulin and shows what appear to be acinar cells producing insulin. The bottom right panel is a confocal image showing colocalization of FITC-labeled anti-insulin and DsRed-labeled anti-amylase, confirming that these are acinar cells. Right panels. Immunoblots of isolated acinar cells after UTMD treatment. The vertical columns are normal control, STZ-treated control, UTMD with betacellulin, UTMD with PDX1, and UTMD with PDX1 and betacellulin. A number of beta-cell markers are upregulated in these UTMD treated acinar cells. Beta-actin is used as a positive control.

FIG. 7. Time course of acinar cell transdifferentiation after UTMD with PDX1 and betacellulin. Top panels. Histological images showing FITC-labeled insulin production in acinar cells, which is prominent at day 10, reduced at day 20, and nearly absent at day 30 after UTMD. Bottom panel. Glucose (left vertical axis) increases between days 10 and 30; whereas insulin (right vertical axis) decreases.

### DETAILED DESCRIPTION OF THE INVENTION

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

The present invention may include modifications and variations of each are possible in light of the teachings described herein without departing from the spirit and scope of the following claims. It is contemplated that the use of the present invention can involve components having different characteristics. It is intended that the scope of the present invention be defined by the claims appended hereto, giving full cognizance to equivalents in all respects.

As used herein, the terms “a sequence essentially as set forth in SEQ ID NO. (#)”, “a sequence similar to”, “nucleotide sequence” and similar terms, with respect to nucleotides, refer to sequences that substantially correspond to any portion of the sequence identified herein as SEQ ID NO.: 1. These terms refer to synthetic as well as naturally-derived molecules and includes sequences that possess biologically, immunologically, experimentally, or otherwise functionally equivalent activity, for instance with respect to hybridization by nucleic acid segments, or the ability to encode all or portions of betacellulin or PDX1 activities. Naturally, these terms are meant to include information in such a sequence as specified by its linear order.

As used herein, the term “gene” refers to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “vector” refers to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

As used herein, the term “host cell” refers to cells that have been engineered to contain nucleic acid segments or altered segments, whether archeal, prokaryotic, or eukaryotic. Thus, engineered, or recombinant cells, are distinguishable from naturally occurring cells that do not contain recombinantly introduced genes through the hand of man.

As used herein, the term “control sequences” refers to DNA sequences necessary for the expression of an operably linked coding sequence in a particular host organism. The control sequences that are suitable for prokaryotes, for example, include a promoter, optionally an operator sequence, a ribosome binding site, and transcriptional terminators. Highly regulated inducible promoters that suppress Fab′ polypeptide synthesis at levels below growth-inhibitory amounts while the cell culture is growing and maturing, for example, during the log phase may be used.

As used herein, the term “operably linked” refers to a functional relationship between a first and a second nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it effects the transcription of the sequence; or a ribosome binding site is operably linked to e coding sequence if it is positioned so as to facilitate translation. Generally, “operably linked” means that the DNA sequences being linked are contiguous and, in the case of a secretory leader, contiguous and in same reading frame. Enhancers do not have to be contiguous. Linking is accomplished by ligation at convenient restriction sites. If such sites do not exist, then synthetic oligonucleotide adaptors or linkers are used in accord with conventional practice.

As used herein, the term “cell” and “cell culture” are used interchangeably end all such designations include progeny. Thus, the words “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Different designations are will be clear from the contextually clear.

As used herein, “Plasmids” are designated by a lower case p preceded and/or followed by capital letters and/or numbers. Starting plasmids may be commercially available, are publicly available on an unrestricted basis, or can be constructed from such available plasmids in accord with published procedures. In addition, other equivalent plasmids are known in the art and will be apparent to the ordinary artisan.

As used herein, the terms “protein”, “polypeptide” or “peptide” refer to compounds comprising amino acids joined via peptide bonds and are used interchangeably.

As used herein, the term “endogenous” refers to a substance the source of which is from within a cell. Endogenous substances are produced by the metabolic activity of a cell. Endogenous substances, however, may nevertheless be produced as a result of manipulation of cellular metabolism to, for example, make the cell express the gene encoding the substance.

As used herein, the term “exogenous” refers to a substance the source of which is external to a cell. When referring to nucleic acids, “exogenous” refers to a nucleic acid sequence that is foreign to the cell, or homologous to the cell but in a position within the host cell nucleic acid in which the element is ordinarily not found. An exogenous substance may nevertheless be internalized by a cell by any one of a variety of metabolic or induced means known to those skilled in the art.

A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed, excised or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ end of the sequences which are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, post-transcriptional cleavage and polyadenylation.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand.

As used herein, the term “transformation,” refers to a process by which exogenous DNA enters and changes a recipient cell, e.g., one or more plasmids that include promoters and coding sequences to express betacellulin and/or PDX1. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome.

As used herein, the term “transfection” refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “vector” is used in reference to nucleic acid molecules that transfer DNA segment(s) from one cell to another. The term “vehicle” is sometimes used interchangeably with “vector.” The term “vector” as used herein also includes expression vectors in reference to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

As used herein, the term “amplify” when used in reference to nucleic acids, refers to the production of a large number of copies of a nucleic acid sequence by any method known in the art. Amplification is a special case of nucleic acid replication involving template specificity. Template specificity is frequently described in terms of “target” specificity. Target sequences are “targets” in the sense that they are sought to be sorted out from other nucleic acid. Amplification techniques have been designed primarily for this sorting out.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, which is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g. ELISA, as well as enzyme-based histochemical assays), fluorescent, radioactive, and luminescent systems. It is not intended that the present invention be limited to any particular detection system or label.

As used herein, the term “target” when used in reference to the polymerase chain reaction, refers to the region of nucleic acid bounded by the primers used for polymerase chain reaction. Thus, the “target” is sought to be sorted out from other nucleic acid sequences. A “segment” is defined as a region of nucleic acid within the target sequence. A target when used in reference to a cell or tissue, refers to the targeting using a vector (e.g., a virus, a liposome or even naked nucleic acids) that are exogenous to a cell to deliver the nucleic acid into the cell such that it changes the function of the cell, e.g., expresses one or more BTC or PDX1 genes.

As used herein, the term “polymerase chain reaction” (“PCR”) refers to the method of K. B. Mullis U.S. Pat. Nos. 4,683,195, 4,683,202, and 4,965,188, hereby incorporated by reference, which describe a method for increasing the concentration of a segment of a target sequence in a mixture of genomic DNA without cloning or purification. This process for amplifying the target sequence includes a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of 32P-labeled deoxynucleotide triphosphates, such as DCTP or DATP, into the amplified segment). In addition to genomic DNA, any oligonucleotide sequence can be amplified with the appropriate set of primer molecules. In particular the amplified segments created by the PCR process itself are, themselves, efficient templates for subsequent PCR amplifications.

As used herein, the term “staining reagent” refers to the overall hybridization pattern of the nucleic acid sequences that comprise the reagent. A staining reagent that is specific for a portion of a genome provides a contrast between the target and non-target chromosomal material. A number of different aberrations may be detected with any desired staining pattern on the portions of the genome detected with one or more colors (a multi-color staining pattern) and/or other indicator methods.

As used herein, the term “transgene” refers to genetic material that may be artificially inserted into a mammalian genome, e.g., a mammalian cell of a living animal. The term “transgenic animal is used herein to describe a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells). Heterologous nucleic acid is introduced into the germ line of such transgenic animals by genetic manipulation of, for example, embryos or embryonic stem cells of the host animal according to methods well known in the art.

As used herein, the term “transgene” refers to such heterologous nucleic acid, e.g., heterologous nucleic acid in the form of, e.g., an expression construct (e.g., for the production of a “knock-in” transgenic animal) or a heterologous nucleic acid that upon insertion within or adjacent a target gene results in a decrease in target gene expression (e.g., for production of a “knock-out” transgenic animal). A “knock-out” of a gene means an alteration in the sequence of the gene that results in a decrease of function of the target gene, preferably such that target gene expression is undetectable or insignificant. Transgenic knock-out animals include a heterozygous knock-out of a target gene, or a homozygous knock-out of a target gene.

As used herein, the term “stem cell” refers to totipotent or pluripotent stem cells, e.g., embryonic stem cells, and to such pluripotent cells in the very early stages of embryonic development, including but not limited to cells in the blastocyst stage of development. In one specific example for use with the present invention, the stem cell may be a pancreatic cell precursor that has not differentiated into an acinar or beta cell and is used as a target to express BTC and/or PDX1.

The present inventors demonstrated that gene therapy could be targeted to pancreatic islets in normal rats, using ultrasound targeted microbubble destruction (UTMD) (6). Intravenous microbubbles carrying plasmid DNA are selectively destroyed within the pancreatic microcirculation by ultrasound, thereby locally delivering the plasmids. Islet specificity was achieved by incorporating the rat insulin-I promoter within the plasmid DNA. It has now been found that using UTMD can be used to deliver betacellulin (BTC), alone and in combination with PDX1 in streptozotocin (STZ)-induced diabetes in rats. Transformation of the target cells led to primitive islet-like clusters of glucagons-staining cells were seen in the rats treated with BTC and PDX1. In this study the clusters disappeared by 30 days after treatment. Although regeneration of normal islets was not seen, diabetes was reversed for up to 15 days after UTMD by transformation of pancreatic acinar cells into insulin-producing cells with beta-cell markers.

Effect of UTMD Gene Therapy on Blood Glucose, Insulin, and C-Peptide. To evaluate the effects of BTC gene therapy on serum glucose and insulin in diabetic rats, 18 rats were treated with intraperitoneal STZ (80 mg/kg) followed UTMD containing a DsRed reporter gene (inactive control, n=6), BTC (n=6), or BTC plus PDX1 (n=6). Six additional controls received STZ alone without UTMD gene therapy (n=3) or neither STZ nor UTMD (normal controls, n=3). Baseline blood glucose was 111±19 mg/dl and was not significantly different between groups.

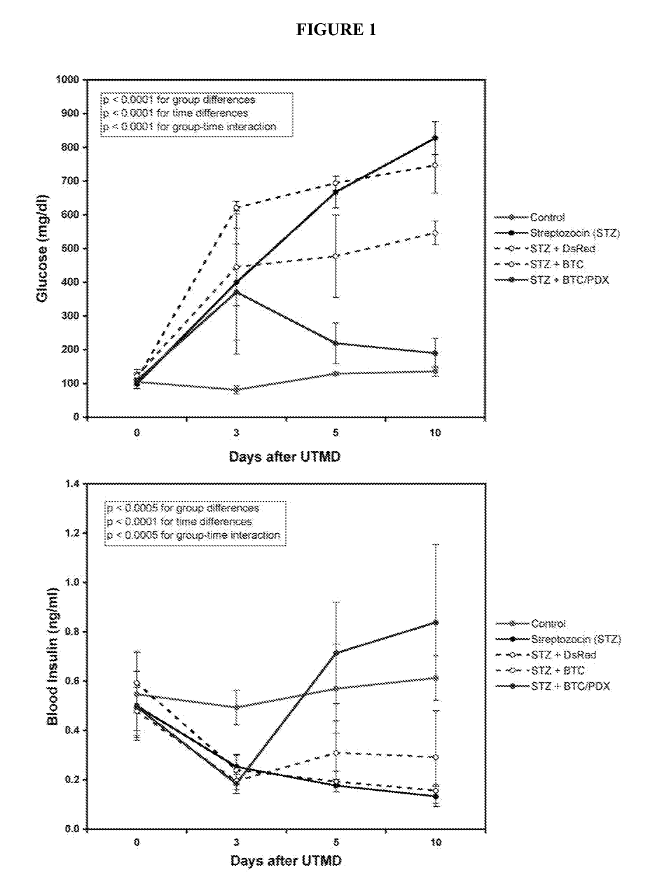

As shown in FIG. 1 (top panel), blood sugar increased dramatically in all STZ-treated rats by day 2 and continued to rise through day 10 in rats treated with STZ alone, the DsRed reporter gene, and BTC alone. However, in rats treated with both BTC and PDX1, glucose decreased from day 3 to day 5, with mean values remaining below 200 mg/dl throughout day 10. By repeated measures ANOVA, differences between treatment groups (F=89.0, p<0.0001) and glucose over time (F=54.7, p<0.0001) were highly statistically significant. By Scheffe post-hoc tests, blood sugar in the rats treated with both BTC and PDX1 was statistically significantly lower than in the STZ alone, DsRed, or BTC groups (p<0.0001), but not statistically different from controls (p=0.17). Blood insulin levels were 0.52±0.11 ng/ml at baseline and were not significantly different between groups.

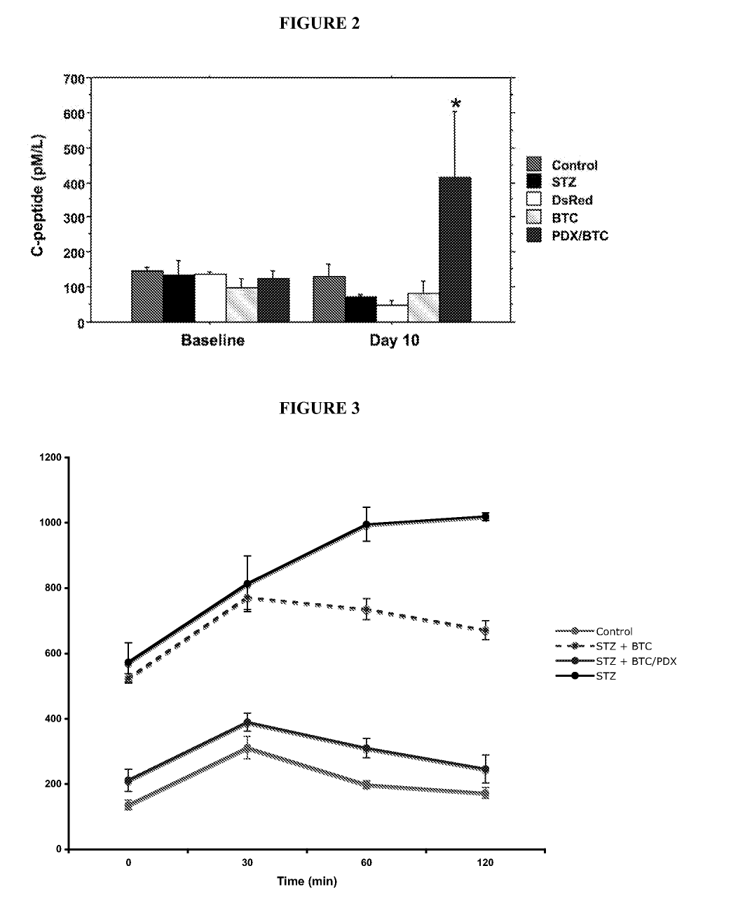

As shown in FIG. 2 (bottom panel), insulin levels decreased at day 3 in all STZ-treated groups relative to normal controls. However, by day 5, insulin levels were higher in the BTC/PDX1 group than in the normal group, although this difference was not statistically significant. By repeated measures ANOVA, differences between treatment groups (F=15.4, p<0.0001) and insulin over time (F=18.9, p<0.0001) were highly statistically significant. It was found, using post-hoc Scheffe tests, that insulin levels were statistically significantly higher in the rats treated with BTC and PDX1 groups compared to those treated with STZ alone (p=0.0087), DsRed (p=0.024), or BTC alone (p=0.015), but not significantly different from controls (p=0.99).

To confirm that the detected insulin was produced by the UTMD treatment, C-peptide levels were measured at baseline and day 10. As shown in FIG. 2, C-peptide remained stable in the normal controls, and decreased in rats treated with STZ alone or STZ with DsRed or BTC. However, C-peptide increased significantly in rats treated with both BTC and PDX1 at day 10. By repeated measures ANOVA, differences in C-peptide between treatment groups was statistically significant (F=10.5, p=0.0004). By post-hoc test, the day 10 C-peptide level was higher in the BTC/PDX1 group compared to all other groups (p<0.03).

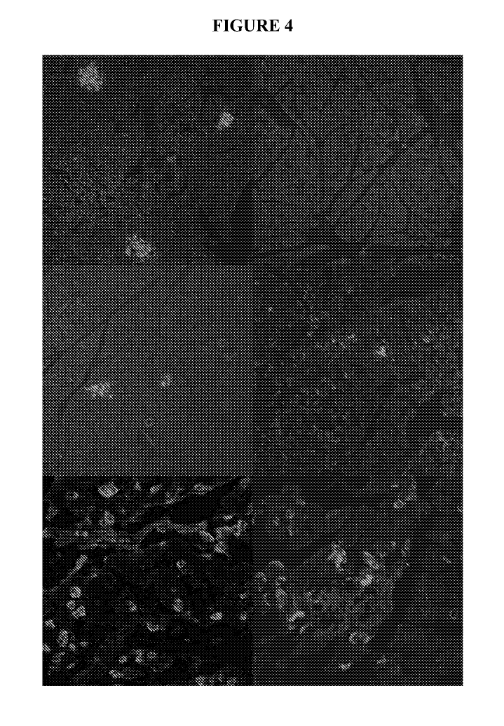

A glucose tolerance test was performed in order to determine whether the UTMD treatments resulted in glucose-regulatable insulin production. As shown in FIG. 3, STZ-treated animals had markedly abnormal glucose tolerance curves, as did rats treated with BTC alone. In contrast, rats treated with BTC and PDX1 had a nearly normal glucose tolerance response.

Immunohistology of Islet Morphology. FIG. 4 shows representative histological samples of rat pancreas at day 10, stained with FITC-labeled anti-insulin (green) and CY5-labeled anti-glucagon (blue). At low power (100×), normal controls (left upper panel) had 3-4 islets per field with central green anti-insulin staining and peripheral blue anti-glucagon staining, consistent with the expected morphology of normal islets. Rats treated with STZ alone (upper right panel) had virtually no detectable anti-insulin staining at day 10, although occasional faint anti-glucagon staining in presumed islet remnants was present. Similar findings were seen in rats treated with DsRed (not shown). In rats treated with BTC alone (left middle panel), 1-2 islet-like clusters were seen per field, mostly consisting of glucagon-positive (blue) cells, with little insulin staining. Rats treated with BTC and PDX1 demonstrated 3-4 islet-like clusters per field, with predominant anti-glucagon staining (right middle panel), shown at high power (400×) in the right lower panel. In addition, there appeared to be substantial anti-insulin staining in the exocrine pancreas at 100×. On higher power (400×, right lower panel), numerous non-islet cells are seen to show cytoplasmic anti-insulin staining.

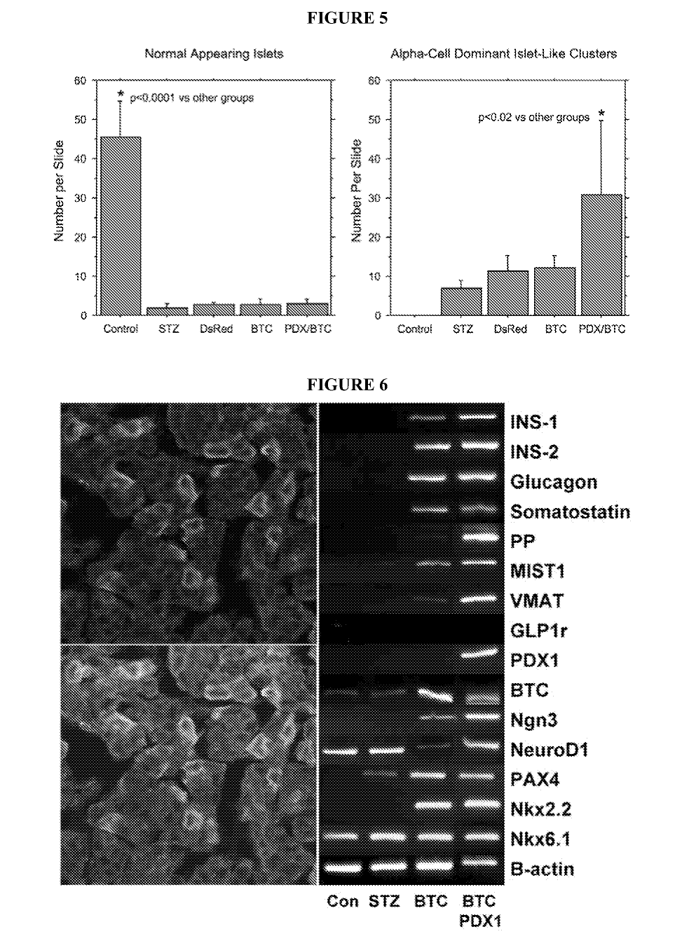

FIG. 5 shows the differences in islet morphology between treatment groups. Normal islets were common in the control group (46±9 islet per slide), but rare in all STZ-treated groups (<3 islets per slide) (p<0.0001 vs control). The unusual islet-like clusters of predominantly glucagons-staining cells were absent in normal controls, being only seen after STZ treatment. These abnormal appearing islets were more prevalent in the rats treated with BTC and PDX1 (19±8 clusters per slide) than in STZ alone (7±2), DsRed (11±4), or BTC alone (12±3), (p<0.02). Moreover, these islet-like clusters of glucagon-staining cells were associated with higher blood glucagons levels in the rats treated with BTC and PDX1 than in other groups. At baseline, blood glucagon was 95±12 pg/ml and no different between groups. At day 10, glucagon had increased to 263±145 pg/ml in the rats treated with BTC and PDX1 (F=4.6, p<0.014), but had not changed significantly in the other groups.

Immunohistology of Insulin-Producing Cells in the Exocrine Pancreas. To further evaluate the insulin-producing cells in exocrine pancreas seen in FIG. 4, confocal microscopy was performed with both FITC-labeled anti-insulin with DsRed-labeled anti-amylase (FIG. 6, left panels). As shown in the top left panel at 1000×, there is prominent expression of insulin, as measured by FITC-conjugate anti-insulin antibodies in the cytoplasm of these cells. The lower left panel shows a confocal image of anti-insulin (green) and anti-amylase (red), in which the signals co-localize, indicating that these are acinar cells. To further confirm that acinar cells were producing insulin, acinar cells were isolated from 4 groups of rats; normal controls, STZ alone, and STZ followed by UTMD with BTC alone, and both BTC and PDX1. The isolated acinar cell fraction was then subjected to RT-PCR for a number of beta-cell markers (FIG. 6, right panel). B-actin, a positive control, was present in all groups, as was Nkx6.1 and neuroD. A number of markers were detected only in the groups receiving BTC and PDX1, including INS-1, INS-2, glucagon, somatostatin, MIST-1, VMAT, neurogenin-3, and Nkx2.2.

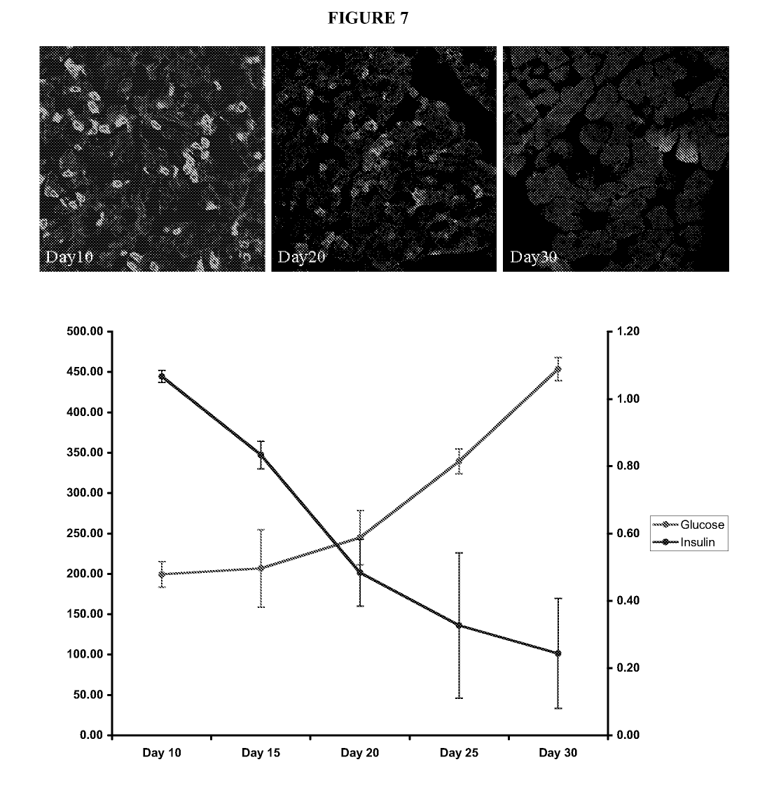

Timecourse of Acinar Cell Production of Insulin. In a separate study, 6 rats were treated with STZ, followed by UTMD with BTC and PDX1, and followed for 30 days. Serum glucose and insulin were measured every 5 days, and 3 rats each were sacrificed at day 20 and 30, respectively. As shown in FIG. 7, the cytoplasmic anti-insulin staining of acinar cells had disappeared by day 30, with marked reduction of serum insulin and elevation of serum glucose. In addition, none of the islet-like clusters of glucagon-positive cells were present at day 30.

Although pharmacological therapy for diabetes has continued to improve, “tight” glucose control has not eliminated the devastating complications of diabetes (7, 8), primarily because available drugs do not reconstitute the glucose-regulatory function of normal islets. Pancreas or islet transplantation can achieve this goal, but are limited by an inadequate donor supply, the need for immunosuppression, and loss of function of the transplanted islets. Thus, current research efforts have focused on creating new sources of beta-cells for transplantation, or regeneration of functioning beta-cells within the pancreas or other tissues (3-5). As disclosed herein, ultrasonic destruction of plasmid-carrying microbubbles was used to direct gene therapy to the pancreas. These data demonstrate that in rats with STZ-induced diabetes, gene therapy with both BTC and PDX1 can restore in vivo insulin production and glucose responsiveness by transformation of pancreatic acinar cells into insulin-producing cells. Although these cells exhibited a number of beta-cell markers, and restored insulin levels and glucose control for up to 15 days, they were absent at 30 days. Thus, the term “transformation” rather than “transdifferentiation,” best described the results using ultrasonic destruction of microbubbles that carry the expression vectors disclosed herein.

The origin of beta-cell mass expansion, which is known to occur after partial pancreatectomy (9), STZ (10), or interferon-gamma (11), has been a subject of recent controversy. Dor, et al. (12) concluded that new islets could only be formed by replication of existing beta-cells after birth or partial pancreatectomy in adult mice. However, a recent study by Hao, et al. (13), showed that isolated human pancreatic epithelial cells could regenerate beta-cells in vitro or when injected along with fetal pancreatic tissue into the renal capsule of immunodeficient mice. The compositions and methods of the present invention were used to stimulate insulin production in the nonendocrine pancreas. Immunohistochemistry was used to localize the insulin-producing acinar cells and the expression of insulin and amylase. Furthermore, when the acinar cell fraction was isolated, it was shown to contain several beta-cell markers in the rats treated with PDX1 and BTC, but not in controls. These observations, along with the fact that gene therapy by UTMD was introduced after complete destruction of existing beta-cells by high dose STZ treatment makes beta-cell replication a highly unlikely explanation for these findings.

UTMD has been shown previously to target genes to the pancreas in vivo, using an insulin promoter to achieve selective expression in islets (4). In the present study, both CMV and rat insulin 1 promoter (RIP) promoters were used because the latter was not expected to work after islet destruction by Streptozotocin (STZ). Instead, it was reasoned that CMV would be useful in initiating beta-cell regeneration in nonendocrine pancreas (14), and that RIP could enhance the process if new beta-cells began to produce insulin. BTC was used alone and in combination with PDX1 to attempt to regenerate beta-cell mass. BTC is a mitogen and beta-cell stimulating hormone (15) that has been shown to induce insulin production in intestinal cells (16) and hepatocytes (17). PDX1 is a transcription factor that is considered a master switch for fetal pancreatic development. Although this combination of genes restored normal insulin and C-peptide levels by transformation of acinar cells into beta-cells, it did not promote regeneration of normal islets. Nevertheless, this study demonstrates that UTMD is a feasible method of introduction of other genes, alone or in various combinations that might be effective in islet regeneration in vivo. This is particularly relevant in the adult animal because genes involved in islet regeneration could differ from those known to regulate embryological development. Potential candidate genes, as reviewed recently by Samson and Chan (4), include INGAP, neurogenin, neuroD, GLP-1, exendin, gastrin and EGF, Mafa, PAX6, Nkx2.2, Nkx6.1, and others.

While acinar cell transformation to a beta-cell phenotype has been reported previously, it was only shown that isolated exocrine cells treated in vitro with EGF and either gastrin or leukemia inhibitory factor, were “transformed” into insulin-producing cells that restored normoglycemia when injected into diabetic rodents. Similarly, infusion of gastrin into rats after ligation of the pancreatic duct, induced beta-cell regeneration with colocalization of amylase and insulin, indicating acinar cell origin (21). In a subsequent study, in alloxan-treated mice the same group found that the beta-cell regeneration originated from ductal cells (22). The present study indicates that acinar cell transformation to a beta-cell phenotype is feasible with restoration of normal insulin production for up to 15 days. It is also demonstrated herein that the cells transformed using the present invention were not due to replication of beta cells or that the cells are of ductal origin. Loss of endocrine function of these acinar cells by 30 days suggests that these cells have not fully “transdifferentiated,” hence the used herein of the term “transformation.” The mechanism by which these cells have lost their insulin-producing capability may relate to the limited duration of effect of the plasmids in vivo. The use of other vectors such as lentivirus or helper-deficient adenovirus can be used instead of plasmids to enhance longevity of the transformation.

Finally, others have been successful in using gene therapy approaches to produce insulin in intestinal or liver cells (17, 23-25). One advantage to the approach taught herein is that the pancreas might be the ideal milieu for islet regeneration. The pancreatic milieu concept is supported by the recent study of Hao et al (13), in which cultured human pancreatic endothelial cells produced islets when injected into the renal capsule of mice along with fetal pancreatic tissue. It is presumed that the fetal pancreatic tissue includes the appropriate stimuli for islet regeneration.

### EXAMPLE 1

Gene therapy with PDX1 and BTC produced primitive islet-like clusters that contained predominantly alpha-cells and disappeared by 30 days. The ability to transform acinar cells into glucose-responsive, insulin producing cells shows for the first time the ability to use UTMD using BTC and PDX1 to regenerate normal islet function. UTMD offers an in vivo non-invasive method for testing candidate genes for islet regeneration in adult animal models of diabetes.

Animal protocol and UTMD. Animal studies were performed in accordance with NIH recommendations and the approval of the institutional animal research committee. Male Sprague-Dawley rats (200 to 250 g) were anesthetized with intraperitoneal ketamine (60 mg/kg) and xylazine (5 mg/kg). Hair was shaved from left abdomen and neck, and a polyethylene tube (PE 50, Becton Dickinson, MD) was inserted into the right internal jugular vein by cut-down. In a first experiment, 24 rats received one of five treatments: 1. No treatment (normal control rats, n=3); 2. STZ (80 mg/kg/ip, Sigma) alone without UTMD (N=3); 3. STZ and UTMD with plasmids encoding a DsRed reporter gene (n=6); 4. STZ and UTMD with plasmids encoding the rat BTC gene (n=6); 5. STZ and UTMD with plasmids encoding both BTC and PDX1 (n=6). For all plasmid preparations, half of the genes contained a RIP promoter and the other half a CMV promoter. Microbubble or control solutions (0.5 ml diluted with 0.5 ml PBS) were infused over 20 minutes via pump (Genie, Kent Scientific).

During the infusion, ultrasound was directed to the pancreas using a commercially available ultrasound transducer (S3, Sonos 5500, Philips Ultrasound, Bothell, Wash.). The probe was clamped in place. Ultrasound was then applied in ultraharmonic mode (transmit 1.3 MHz/receive 3.6 MHz) at a mechanical index of 1.4. Four bursts of ultrasound were triggered to every fourth end-systole by ECG using a delay of 45-70 ms after the peak of the R wave. These settings have shown to be optimal for plasmid delivery by UTMD using this instrument (26). Bubble destruction was visually apparent in all rats. After UTMD, the jugular vein was tied off, the skin closed, and the animals allowed to recover. Blood samples were drawn after an overnight 10 hour fast at baseline, and at 3, 5, and 10 days after treatment. Animals were sacrificed at day 10 using an overdose of sodium pentobarbital (120 mg/kg). Pancreas, liver, spleen and kidney were harvested for histology and assessment of PDX1 and betacellulin proteins by Western blot. Blood glucose level was measured with blood glucose test strip (Precision, Abbott), blood insulin, C-peptide and glucagons level were measured with RIA kit from Linco Research.

In a second protocol designed to assess time course of acinar cell transdifferentiation, 6 rats with STZ-induced diabetes were treated with UTMD using microbubbles containing both PDX1 and betacellulin plasmids. Blood samples for glucose, insulin, C-peptide, and glucagons were obtained at days 5, 10, 15, 20, 25, and 30 after UTMD. Half the rats were sacrificed at day 20 for histology (day 25 and 30 blood samples not obtained in these), and the other half sacrificed at day 30.

### EXAMPLE 2

Manufacture of Plasmid-Containing Lipid-Stabilized Microbubbles. Lipid-stabilized microbubbles were prepared as previously described by the inventors (6). Briefly, 250 μl of DPPC (1,2-dipalmitoyl-sn-glycero-3-phosphatidylcholine, Sigma, St. Louis, Mo.) 2.5 mg/ml and DPPE (1,2-dipalmitoyl-sn-glycero-3-phosphatidylethanolamine, Sigma, St. Louis, Mo.) 0.5 mg/ml solution and 50 μl of pure glycerol was added to 1.5 ml vials containing 2 mg of dried plasmid and mixed well and 20 μg of dexamathasone and incubated on room temperature for 30 min. the remaining headspace was filled with the perfluoropropane gas (Air Products, Inc, Allentown, Pa.) and then mechanically shaken for 30 seconds by a dental amalgamator (Vialmix™, Bristol-Myers Squibb Medical Imaging, N. Billerica, Mass.). The mean diameter and concentration of the microbubbles were measured by a particle counter (Beckman Coulter Multisizer III).

Plasmid Constructs. Rat insulin 1 promoter (RIP) fragment (from −412 to +165; genbank#: J00747) was PCR amplified from Sprague-Dawley DNA by using following PCR primers:

| | |
|---|---|
| primer 1 (XhoI) | |
| 5′-CAACTCGAGGCTGAGCTAAGAATCCAG-3′; | (SEQ ID NO.: 1) |
| primer 2 (EcoRI) | |
| 5′-GCAGAATTCCTGCTTGCTGATGGTCTA-3′. | (SEQ ID NO.: 2) |

Following rat PDX1 and betacellulin cDNA preparation: New born rat pancreatic samples were flash frozen in liquid nitrogen and stored at −86° C. The frozen samples were thawed in 1 ml of RNA-STAT solution and immediately homogenized using a polytron 3000 homogenizer at 10,000 rpm for 30 s two times. Total RNA was prepared from the specimens using an RNeasy Mini Kit (QIAGEN) according to the manufacturer's instructions. RNA (30 ng) was reverse-transcribed in 20 μl by using a Sensiscript RT Kit (QIAGEN) with oligo(dT)₁₆. The reaction mixture was incubated at 42° C. for 50 min, followed by a further incubation at 70° C. for 15 min. PCR was performed for all samples using a GeneAmp PCR System 9700 (PE ABI) in 50 μl volume containing 2 μl cDNA, 25 μl of HotStarTaq Master Mix (QIAGEN) and 20 pmol of each primer:

Rat PDX1 cDNA primer (from Genbank#: NM022852):

(SEQ ID NO.: 3)

5′AAGAATTCCCATGAATAGTGAGGAGCA3′(sense);

(SEQ ID NO.: 4)

5′AAGCGGCCGC TCAGCCTGCGGTCCTCACC3′(antisense).
Rat betacellulin cDNA primers (from Genbank#:
NM022256):

(SEQ ID NO.: 5)

5′AAGAATTCCGGTTGATGGACTCACT3′(sense);

(SEQ ID NO.: 6)

5′AAGCGGCCGCCATTAAGTTAAGCAATAT(antisense).

The corresponding PCR products were purified by agarose gel electrophoresis and QIAquick Gel Extraction kit (QIAGEN). PCR products were sequenced with dRhodamine Terminator Cycle Sequencing Kit (PE Applied Biosystems, Foster City, Calif.) on an ABI 3100 Genomic Analyzer. RIP fragments were digested with XhoI and EcoRI and then ligated into the XhoI-EcoRI sites of pDsRed-Express-1 vector (BD Biosciences). PDX1 and betacellulin cDNA fragments digested with EcoR1 and Not1 and ligated into the EcoR1 and Not1 sites of RIP or CMV driving vectors and ligation reactions were carried out in 20 ml of 20 mM tris-HCL, 0.5 mMATP, 2 mM dithiothreitol and 1 unit of T4 DNA ligase. Cloning and isolation of plasmid were performed by standard procedures.

Isolation of acinar cells and RT-PCR. Acinar cells were isolated as previously described {10}. In brief, 1 gram of rat pancreas tissue was placed in a 100 mL beaker with 20 mL RPMI-1640 medium containing 200 U/ml collagenase, 10 mM Hepes, 5% fetal bovine serum, penicillin 100 U/mL, streptomycin 50 μg/mL, and soybean trypsin inhibitor 0.2 mg/mL. It was cut into very small pieces with a scissor and transferred to a sterile flask, and incubated at 37° C. with reciprocal shaking at 150 cycles/min for 40 min. Acinar cells suspension were filtered with 100 μm mesh nylon gauze. The acinar cells were cultured with RPMI-1640 medium containing 10% FBA and 4 mM streptozocin (depleted of residual beta cells) at 37° C., 5% CO₂ for 2 hrs. The cells were harvested and total RNA were extracted and reversed into cDNA pool. PCR was performed for all samples using a GeneAmp PCR System 9700 (PE ABI) in 25 μl volume containing 1 μl cDNA, 12.5 μl of HotStarTaq Master Mix (QIAGEN) and 20 pmol of each prime. PCR products were confirmed by sequencing. Table 1 includes examples of pairs of PCR oligos.

TABLE 1
PCR Oligos

| | Forward; | Reverse, |
|---|---|---|
| Name | SEQ ID NO.: | SEQ ID NO.: |
| Rat insulin-1 | ATGGCCCTGTGG | CAGTGCCAAGGT |
| | ATCCGCTT; 7 | CTGAAGGT; 8 |
| Rat insulin-2 | ATGGCCCTGTGG | CCAGTTGGTAGA |
| | ATGCGCTT; 9 | GGGAGCAG; 10 |
| Rat glucagons | ATGAAGACCGTT | CAGCTATGGCGA |
| | TACATCGT; 11 | CTTCTTCC; 12 |
| Rat PP | ATGGCCGTCGCA | TCAGCTCCGGGCA |
| | TACTACTG; 13 | GCAGCGCA; 14 |
| Rat Somatostatin | ATGCTGTCCTGC | GAAGTTCTTGCAG |
| | CGTCTCCA; 15 | CCAGCTT; 16 |
| Rat GLP1r | ATCCACCTGAAC | ATGACCCGGATGA |
| | CTGTTTG; 17 | AGACAA; 18 |
| Rat VMAT | AGACCATGTGTT | CGAAGGAAAAAGC |
| | CCCGAAA; 19 | AGAGTG; 20 |
| Rat Mist1 | GAAGTGACCAAG | CTCCCCTCTCTGA |
| | GGTCTTC; 21 | AGCTGTG; 22 |
| Rat BTC | ATGGACTCGACT | CATGACGCCTATC |
| | GCCCCAGG; 23 | AAGCAGA; 24 |
| Rat pdx1 | TTCCCGAATGGA | GTTACGGCACAAT |
| | ACCGAGAC; 25 | CCTGCTC; 26 |
| Rat NEUROG3 | CACGAAGTGCTC | AGGCTACCAGCTT |
| | AGTTCCAA; 27 | GGGAAAC; 28 |
| Rat NEUROD1 | GGATGATCAAAA | TGCAGGGTAGTGC |
| | GCCCAAGA; 29 | ATGGTAA; 30 |
| Rat PAX4 | ATGGCGCAGACA | ATAGGTTGATGGC |
| | AGAGAAGT; 31 | GCTTGTC; 32 |
| Rat PAX6 | TGTCCAACGGAT | TTGGTGTTTTCTC |
| | GTGTGAGT; 33 | CCTGTCC; 34 |
| Rat NKX2.2 | GTCGCTGACCAA | CAGTCCGTGCAGG |
| | CACAAAGA; 35 | GAGTATT; 36 |
| Rat NKX6.1 | AGACCCACATTC | TCCAGGGGCTTGT |
| | TCCGGCCA; 37 | TGTAATC; 38 |

Immunohistochemistry. Cryostat sections 5 μm in thickness were fixed in 4% paraformaldehyde for 15 min at 4° C. and quenched for 5 min with 10 mM glycine in PBS. Sections were then rinsed in PBS 3 times, and permeabilized with 0.2% Triton X-100 in PBS for 10 min. Sections were blocked with 10% goat serum and 10% bovine serum at 37° C. for 1 hr and washed with PBS 3 times. The primary antibody (anti-mouse insulin, 1:5000 dilution from Sigma; anti-rabbit glucagon, 1:500 dilution, Chemicon; anti-rabbit pdx1 and anti-rabbit betacellulin, 1:500 dilution, Chemicon Co; anti-alpha amylase, 1:500 dilution, Abcam) was added and incubated at 4° C. overnight. After washing with PBS three times for 5 min, the secondary antibody (anti-mouse IgG conjugated with FITC, 1:250 dilution, Sigma Co., anti-rabbit IgG conjugated with Cy5, 1:250 dilution, Chemicon) was added and incubated for 1 hr at 37° C. Sections were rinsed with PBS for 10 min, 5 times, and then mounted. Confocal microscopy was used to detect FITC signal (488 nm/510 nm) and Cy5 signal (633 nm/710 nm).

Data analysis. Data was analyzed with Statview software (SAS, Cary, N.C.). The results are expressed as mean ±one standard deviation. Differences were analyzed by repeated measures ANOVA with Fisher's post-hoc test and considered significant at p<0.05.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Wild S, Roglic G, Green A, Sicree R, King H. Global prevalence of diabetes: estimates for the year 2000 and projections for 2030. Diabetes Care 2004; 27:1047-53.
2. Roglic G, Unwin N, Bennett P H, Mathers C, Tuomilehto J, Nag S, Connolly V, King H. The burden of mortality attributable to diabetes: realistic estimates for the year 2000. Diabetes Care 2005; 28:2130-35.
3. Bonner-Weir S, Wier G C. New sources of pancreatic β-cells. Nature Biotech 2005; 23:857-61.
4. Samson S, Chan L. Gene therapy for diabetes: reinventing the islet. Trends in Endocrinology and Metabolism 2005; 17:92-100.
5. Lipsett M, Aikin R, Castellarin M, Hanley S, Jamal A, Laganiere S, Rosenberg L. Islet Neogenesis: A potential therapeutic tool in type 1 diabetes. Int J Biochem Cell Biol. 2006; 38:498-503.
6. Chen S, Ding J H, Bekeredjian R, Yang B Z, Shohet R V, Johnston S A, Hohmeier H E, Newgard C B, Grayburn P A. Efficient gene delivery to pancreatic islets with ultrasonic microbubble destruction technology. Proc Natl Acad Sci USA. 2006; 103:8469-74.
7. The DCCT Research Group. The effect of intensive treatment of diabetes on the development and progression of long-term complications in insulin-dependent diabetes mellitus. N Engl J Med 1993; 329:977-986.
8. The EDIC Study Group. Sustained effect of intensive treatment in type I diabetes mellitus on development and progression of diabetic retinopathy: The Epidemiology of Diabetes Interventions and Complications (EDIC) study. JAMA 2003; 290:2159-2167.
9. Bonner-Weir S, Baxter L A, Schuppin G T, Smith F E. A second pathway for regeneration of adult exocrine and endocrine pancreas. A possible recapitulation of embryonic development. Diabetes 1993; 42:1715-1720.
10. Guz Y, Nasir I, Teitelman G. Regeneration of pancreatic beta cells from intra-islet precursor cells in an experimental model of diabetes. Endocrinology 2001; 142: 4956-4968.
11. Sarvetnick N E, Gu D. Regeneration of pancreatic endocrine cells in interferon-gamma transgenic mice. Adv Exp Med Biol 1992; 321:85-89.
12. Dor Y, Brown J, Martinez 01, Melton D A. Adult pancreatic beta-cells are formed by self-duplication rather than stem cell differentiation. Nature 2004; 429:41-46.
13. Hao E, Tyrberg B, Itkin-Ansari P, Lakey J, Geron I, Monosov E Z, Barcova M, Mercola M, Levine F. Beta-cell differentiation from nonendocrine epithelial cells of the adult human pancreas. Nature Medicine 2006; 12:310-316.
14. Zhan Y, Brady J L, Johnston A M, Lew A M. Predominate Transgene Expression in Exocrine Pancreas Directed by the CMV Promoter. DNA and Cell Biology 2000; 19: 639-645.
15. Shing Y, et al. Betacellulin: a mitogen from pancreatic beta cell tumors. Science 1993; 259:2681-2689.
16. Kojima H, et al. Combined expression of pancreatic duodenal homeobox 1 and islet factor 1 induces immature enterocytes to produce insulin. Diabetes 2002; 51:1398-1408.
17. Kojima H, Fujimaya M, Matsumura K, Younan P, Imaeda H, Maeda M, Chan L. NeuroD-betacellulin gene therapy induces islet neogenesis in the liver and reverses diabetes in mice. Nature Medicine 2003; 9:596-603.
18. Lardon, J., Huyens, N., Rooman, I., Bouwens, L. Exocrine cell transdifferentiation in dexamethasone-treated rat pancreas. Virchows Arch. 2004; 444:61-65.
19. Baeyens L, De Breuck S, Lardon J, Mfopou J K, Rooman I, Bouwens L. In vitro generation of insulin-producing beta cells from adult exocrine pancreatic cells. Diabetologia 2005; 48:49-57.
20. Rooman I., Heremans Y., Heimberg H., Bouwens L. Modulation of rat pancreatic acinoductal transdifferentiation and expression of PDX-1 in vitro. Diabetologia 2000; 43: 907-914.
21. Rooman, I. et al. Mitogenic effect of gastrin and expression of gastrin receptors in duct-like cells of rat pancreas. Gastroenterology 2001; 121:940-949.
22. Rooman, I. & Bouwens, L. Combined gastrin and epidermal growth factor treatment induces islet regeneration and restores normoglycemia in C57BL/6J mice treated with alloxan. Diabetologia 2004; 47:259-265.
23. Ferber S, Halkin A, Cohen H, Ber I, Einav Y, Goldberg I, Barshack I, Seijffers R, Kopolovic J, Kaiser N, Karasik A. Pancreatic and duodenal homeobox gene 1 induces expression of insulin genes in liver and ameliorates streptozotocin-induced hyperglycemia. Nature Medicine 2000; 6:568-572.
24. Fujita, Y., Cheung, A. T., & Kieffer, T. J. Harnessing the gut to treat diabetes. Pediatr. Diabetes (suppl. 2) 2004; 5: 57-59.
25. Kaneto H, Matsuoka T, Nakatani Y, Miyatsuka T, Matsuhisa M, Hori M, Yamasaki Y. A crucial role of MafA as a novel therapeutic target for diabetes. J. Biol. Chem. 2005; 280:15047-15052.
26. Chen S Y, Shohet R V, Bekeredjian R, Frenkel P A, Grayburn P A. Optimization of ultrasound parameters for cardiac gene delivery of adenoviral and plasmid deoxyribonucleic acid by ultrasound-targeted microbubble destruction. J Am Coll Cardiol 2003; 42:301-308.

What is claimed is:

1. A method for inducing insulin production in cells, comprising:
transforming one or more cells with a vector that expresses betacellulin (BTC) and Pancreas Duodenum Homeobox-1 (PDX1).

2. The method of claim 1, wherein the cells are transformed with a single construct that co-expresses BTC and PDX1.

3. The method of claim 1, wherein the cells are selected from pancreatic islet cells, pancreatic acinar cells, cell lines, cells that have been co-transfected with one or more insulin genes.

4. The method of claim 1, wherein the construct is delivered using microbubbles, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

5. The method of claim 1, wherein the construct is delivered using microbubbles and ultrasound targeted microbubble destruction.

6. The method of claim 1, wherein the cells express insulin based on glucose levels for more than 15 days.

7. The method of claim 1, wherein the cells are transformed in vivo and express insulin in a glucose responsive manner for more than 15 days.

8. The method of claim 1, wherein the cells are nonendocrine pancreas cells are express insulin in a glucose responsive manner for more than 15 days.

9. The method of claim 1, wherein the BTC, the PDX1, or both are selected from mouse, rat or human.

10. A vector comprising a nucleic acid expression construct that expresses betacellulin, PDX1 or both when transfected into acinar cells.

11. The vector of claim 10, wherein the vector is disposed with a microbubble that is destroyed upon exposure to ultrasound.

12. The vector of claim 10, wherein the nucleic acid expression construct comprises a promoter the controls the expression of BTC.

13. The vector of claim 10, wherein the nucleic acid expression construct comprises BTC and PDX1 under the control of the same promoter.

14. The vector of claim 13, wherein the BTC and PDX1 are on separate vectors.

15. The vector of claim 13, wherein the BTC and PDX1 are under the control of different promoters.

16. The vector of claim 13, wherein the promoter is selected from RIP, HIV, AMV, SV40, Mouse Mammary Tumor Virus (MMTV) promoter, Human Immunodeficiency Virus Long Terminal Repeat (HIV LTR) promoter, Moloney virus promoter, ALV promoter, Cytomegalovirus (CMV) promoter, human Actin promoter, human Myosin promoter, RSV promoter, human Hemoglobin promoter, human muscle creatine promoter and EBV promoter.

17. The vector of claim 10, wherein the wherein the BTC, the PDX1, or both are selected from mouse, rat or human.

18. The vector of claim 10, wherein the PDX1 is Genbank Accession No. NM022852 and the BTC is Genbank Accession No. NM022256.

19. A host cell comprising an exogenous nucleic acid segment that expresses BTC and PDX1 under the control of a constitutive promoter.

20. The host cell of claim 19, wherein the promoter is selected from RIP, HIV, AMV, SV40, Mouse Mammary Tumor Virus (MMTV) promoter, Human Immunodeficiency Virus Long Terminal Repeat (HIV LTR) promoter, Moloney virus promoter, ALV promoter, Cytomegalovirus (CMV) promoter, human Actin promoter, human Myosin promoter, RSV promoter, human Hemoglobin promoter, human muscle creatine promoter and EBV promoter.

21. The host cell of claim 19, wherein the BTC, the PDX1, or both are selected from mouse, rat or human.

22. The host cell of claim 19, wherein the host cell is selected from pancreatic islet cells, pancreatic acinar cells, primary pancreatic cells, or cells that have been co-transfected with one or more insulin genes.

23. The host cell of claim 19, wherein the host cell is transformed by microbubbles, calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

24. The host cell of claim 19, wherein the cells express pancreatic beta cell markers selected from INS-1, INS-2, glucagon, somatostatin, MIST-1, VMAT, neurogenin-3, Nkx2.2 and combinations thereof.


SEQUENCE LISTING (38 sequences)

SEQ ID NO: 1 - DNA, 27 bp, Artificial Sequence
  Chemically Synthesized Oligonucleotides
caactcgaggctgagctaagaatccag

SEQ ID NO: 2 - DNA, 27 bp, Artificial Sequence
  Chemically Synthesized Oligonucleotides
gcagaattcctgcttgctgatggtcta

SEQ ID NO: 3 - DNA, 27 bp, Artificial Sequence
  Chemically Synthesized Oligonucleotides
aagaattcccatgaatagtgaggagca

SEQ ID NO: 4 - DNA, 29 bp, Artificial Sequence
  Chemically Synthesized Oligonucleotides
aagcggccgctcagcctgcggtcctcacc

SEQ ID NO: 5 - DNA, 25 bp, Artificial Sequence
  Chemically Synthesized Oligonucleotides
aagaattccggttgatggactcact

SEQ ID NO: 6 - DNA, 28 bp, Artificial Sequence
  Chemically Synthesized Oligonucleotides
aagcggccgccattaagttaagcaatat

SEQ ID NO: 7 - DNA, 20 bp, Artificial Sequence
  Chemically Synthesized Oligonucleotides
atggccctgtggatccgctt

SEQ ID NO: 8 - DNA, 20 bp, Artificial Sequence
  Chemically Synthesized Oligonucleotides
cagtgccaaggtctgaaggt

SEQ ID NO: 9 - DNA, 20 bp, Artificial Sequence
  Chemically Synthesized Oligonucleotides
atggccctgtggatgcgctt

SEQ ID NO: 10 - DNA, 20 bp, Artificial Sequence
  Chemically Synthesized Oligonucleotides
ccagttggtagagggagcag

SEQ ID NO: 11 - DNA, 20 bp, Artificial Sequence
  Chemically Synthesized Oligonucleotides
atgaagaccgtttacatcgt

SEQ ID NO: 12 - DNA, 20 bp, Artificial Sequence
  Chemically Synthesized Oligonucleotides
cagctatggcgacttcttcc

SEQ ID NO: 13 - DNA, 20 bp, Artificial Sequence
  Chemically Synthesized Oligonucleotides
atggccgtcgcatactactg

SEQ ID NO: 14 - DNA, 21 bp, Artificial Sequence
  Chemically Synthesized Oligonucleotides
tcagctccgggcagcagcgca

SEQ ID NO: 15 - DNA, 20 bp, Artificial Sequence
  Chemically Synthesized Oligonucleotides
atgctgtcctgccgtctcca

SEQ ID NO: 16 - DNA, 20 bp, Artificial Sequence
  Chemically Synthesized Oligonucleotides
gaagttcttgcagccagctt

SEQ ID NO: 17 - DNA, 19 bp, Artificial Sequence
  Chemically Synthesized Oligonucleotides
atccacctgaacctgtttg

SEQ ID NO: 18 - DNA, 19 bp, Artificial Sequence
  Chemically Synthesized Oligonucleotides
atgacccggatgaagacaa

SEQ ID NO: 19 - DNA, 19 bp, Artificial Sequence
  Chemically Synthesized Oligonucleotides
agaccatgtgttcccgaaa

SEQ ID NO: 20 - DNA, 19 bp, Artificial Sequence
  Chemically Synthesized Oligonucleotides
cgaaggaaaaagcagagtg

SEQ ID NO: 21 - DNA, 19 bp, Artificial Sequence
  Chemically Synthesized Oligonucleotides
gaagtgaccaagggtcttc

SEQ ID NO: 22 - DNA, 20 bp, Artificial Sequence
  Chemically Synthesized Oligonucleotides
ctcccctctctgaagctgtg

SEQ ID NO: 23 - DNA, 20 bp, Artificial Sequence
  Chemically Synthesized Oligonucleotides
atggactcgactgccccagg

SEQ ID NO: 24 - DNA, 20 bp, Artificial Sequence
  Chemically Synthesized Oligonucleotides
catgacgcctatcaagcaga

SEQ ID NO: 25 - DNA, 20 bp, Artificial Sequence
  Chemically Synthesized Oligonucleotides
ttcccgaatggaaccgagac

SEQ ID NO: 26 - DNA, 20 bp, Artificial Sequence
  Chemically Synthesized Oligonucleotides
gttacggcacaatcctgctc

SEQ ID NO: 27 - DNA, 20 bp, Artificial Sequence
  Chemically Synthesized Oligonucleotides
cacgaagtgctcagttccaa

SEQ ID NO: 28 - DNA, 20 bp, Artificial Sequence
  Chemically Synthesized Oligonucleotides
aggctaccagcttgggaaac

SEQ ID NO: 29 - DNA, 20 bp, Artificial Sequence
  Chemically Synthesized Oligonucleotides
ggatgatcaaaagcccaaga

SEQ ID NO: 30 - DNA, 20 bp, Artificial Sequence
  Chemically Synthesized Oligonucleotides
tgcagggtagtgcatggtaa

SEQ ID NO: 31 - DNA, 20 bp, Artificial Sequence
  Chemically Synthesized Oligonucleotides
atggcgcagacaagagaagt

SEQ ID NO: 32 - DNA, 20 bp, Artificial Sequence
  Chemically Synthesized Oligonucleotides
ataggttgatggcgcttgtc

SEQ ID NO: 33 - DNA, 20 bp, Artificial Sequence
  Chemically Synthesized Oligonucleotides
tgtccaacggatgtgtgagt

SEQ ID NO: 34 - DNA, 20 bp, Artificial Sequence
  Chemically Synthesized Oligonucleotides
ttggtgttttctccctgtcc

SEQ ID NO: 35 - DNA, 20 bp, Artificial Sequence
  Chemically Synthesized Oligonucleotides
gtcgctgaccaacacaaaga

SEQ ID NO: 36 - DNA, 20 bp, Artificial Sequence
  Chemically Synthesized Oligonucleotides
cagtccgtgcagggagtatt

SEQ ID NO: 37 - DNA, 20 bp, Artificial Sequence
  Chemically Synthesized Oligonucleotides
agacccacattctccggcca

SEQ ID NO: 38 - DNA, 20 bp, Artificial Sequence
  Chemically Synthesized Oligonucleotides
tccaggggcttgttgtaatc